(19) United States
(12) Patent Application Publication
(10) Pub. No.: US 2025/0107935 A1
(43) Pub. Date: Apr. 3, 2025

(54) WOUND DRESSING

(71) Applicant: KERECIS LIMITED, Isafjordur (IS)

(72) Inventors: Gudmundur Fertram SIGURJONSSON, Isafjordur (IS); Gunnar JOHANNSSON, Isafjordur (IS); Dora Hlin GISLADOTTIR, Isafjordur (IS); Dan MOORADIAN, Isafjordur (IS)

(21) Appl. No.: 18/901,619

(22) Filed: Sep. 30, 2024

Related U.S. Application Data
(60) Provisional application No. 63/541,661, filed on Sep. 29, 2023.

(51) Int. Cl. A61F 13/0203

(52) U.S. Cl. CPC A61F 13/0223

(57) ABSTRACT

An integrated wound treatment with a skin substitute is provided for direct contact with a polymeric contact layer. The polymeric contact layer provides sufficient tackiness to negate the need for an adhesive between the polymeric contact layer and the skin substitute. The wound treatment is arranged to be submersed within a saline solution to allow for hydration of the skin substitute, together with the polymeric contact layer, without disrupting the tackiness or adhesion properties of the polymeric contact layer.

### FIELD OF THE DISCLOSURE

The disclosure relates generally to wound treatments for stabilizing, protecting, and/or healing damaged tissue and methods for making the same.

### BACKGROUND

The skin is the largest organ in the human body and acts as a protective barrier against physical injury (e.g., wounds) and foreign bodies. A wound is defined as a breakage in the continuity of the skin. When the skin is damaged, microorganisms can contaminate and infect the wound. Wounds can be classified into two types: acute and chronic. Acute wounds heal normally efficiently and follow the natural phases of hemostasis, inflammation, proliferation, and remodeling. Chronic wounds do not follow these natural phases and instead remain inflamed, often getting “stalled” in one of the natural phases of wound healing. If left untreated, chronic wounds can develop and even lead to fatal complications.

Wound treatments and dressings should utilize the natural principles of wound healing. Namely, the wound treatment should provide a moist environment to optimize healing. However, a moist, warm environment created by wounds, e.g., ulcers, also provides an ideal environment for bacterial growth. A large amount of drainage can indicate infection, whereas a reduction in drainage can indicate inadequate arterial circulation or that an infection is resolving. Thus, proper drainage of wound exudate is needed to promote effective healing.

Existing wound dressings can be classified in numerous ways depending on functionality in the wound, type of material, and the physical form of the dressing. Foam dressings (e.g., Lyofoam™, Allevyn™, Tielle™) provide adequate absorbency and thermal insulation, but their strong adhesive properties can cause dermatitis. Low-adherence dressings (e.g., Melolin™, Mepore™, and Mepitel™) are low-cost and typically hypoallergenic; however, these dressings have minimal absorptive capacity and are unsuited for all but lightly exudating and superficial wounds. Silver-impregnated dressings exhibit broad antibacterial properties, but there are disputable impacts, e.g., limited by hypersensitivity, and increased manufacturing costs. Hydrogels (e.g., Intrasite™, Nu-gel™, Aquaform™) have a gel-like nature and consist of starch polymers with increased water's intrinsic content. Hydrogels are primarily used to donate fluid to dry necrotic wounds; however, their absorbency is limited, they require tape for fixation, and they are unsuitable for oozing exudate because of their variable or altered porosity. Thus, there is a need for an improved dressing that (1) provides sufficient tackiness to remain fixed in place at the wound site without causing dermatitis, (2) is hypoallergenic, (3) is cost-effective, and (4) avoids separate adhesives or tapes for fixation.

Regarding another principle of wound healing, it is desirable for the dressing or treatment to aid in managing the infection. The field of regenerative medicine focuses on the growth and replacement of damaged tissue by facilitating the growth and proliferation of host cells at the site of injury, resulting in faster healing time and a more permanent solution. Various human, animal, and synthetic materials are currently described or used in medical procedures to augment, repair, or correct tissue defects.

For example, the Integra® Dermal Regeneration Template (“Integra Template”) has two layers: a thin outer layer of silicone and a thick inner matrix layer of pure bovine collagen and glycosaminoglycan (GAG). Both collagen and GAG are normal components of human skin. The collagen is obtained from bovine tendon collagen, and the glycosaminoglycan is obtained from shark cartilage. However, the Integra Template is limited by its relatively high cost and the need for a second operation to place a skin graft over the new dermis.

The Integra Template requires handling of multiple cover sheets, including the removal of a first cover sheet followed by a second cover sheet before being placed in a sterile saline solution, separate shaping of the Integra Template to fit the wound, and separate staples or sutures to hold the Integra Template in place. This process delays the application of the template to the wound and increases the chance of product misplacement. Additionally, because the silicone layer is separately applied, there exists the potential complication of applying the layer upside down and thereby missing the critical step of applying the collagen template layer directly to the wound.

U.S. Published Patent Application No. 2003/0059460, filed Sep. 24, 2022, discloses a hybrid polymer material comprising synthetic and natural polymers that can regenerate living body tissue. The hybrid material comprises a cross-linked naturally occurring polymer and a biodegradation-absorbable synthetic polymer. However, a series of complicated process steps must be undertaken to produce the hybrid material. In addition, the resulting hybrid material contains synthetic and naturally occurring materials.

Additionally, U.S. Pat. No. 8,613,957, granted Dec. 24, 2013, and incorporated herein by reference, describes an exemplary scaffold material for wound care and other tissue healing applications constituted of a decellularized extracellular matrix from fish skin. The decellularized fish skin product provides an intact scaffold to support endothelial and epithelial cell ingrowth. The decellularized fish skin scaffold material is also biocompatible and thus can be integrated by the host. MariGen™ is a commercially available skin substitute made from the minimally processed skin of wild-caught Atlantic cod originating from Iceland. The fish skin is structurally similar to human skin, with three basic layers: epidermis, dermis, and hypodermis. It contains proteins, lipids, fatty acids, and other bioactive compounds homologous to human skin. MariGen™ is used to manage chronic wounds such as diabetic wounds, pressure ulcers, vascular ulcers, and draining wounds commonly treated in private offices and wound care centers.

The inventors of the present disclosure discovered that the assembly of both an acellular matrix, similar to the kind used in MariGen™, and a separate fixation or securing layer is problematic. Handling multiple components separately of an acellular matrix and securing layer creates more opportunities for unsanitary conditions and treatment. There is a need for an integrated, aseptic solution that allows for simplified handling and processing of regenerative wound treatment.

In particular, there is a need for an integrated, aseptic wound treatment that (1) protects the wound and skin substitute, (2) expedites the application process to the wound, and (3) simplifies manufacturing and inventory issues. The wound treatment described in the present disclosure satisfies these needs and provides numerous advantages over the prior art.

### SUMMARY

The present disclosure is directed to an improved, integrated wound treatment for supporting tissue regeneration of wounds. The wound treatment comprises a skin substitute, an integrated, gently adherent polymeric contact layer, and a release liner. The polymeric contact layer preferably comprises an elastomeric layer and a hydrophobic membrane. The release liner is disposed over and in direct contact with the skin substitute and the elastomeric layer of the polymer contact later.

The skin substitute is conveniently arranged between the polymer contact later and the release liner. The skin substitute is arranged to be submersed in a saline solution, together with the polymeric contact layer and release liner, for hydration of the skin substitute. Such capability simplifies preparations of the wound treatment for application to the wound.

The skin substitute is selected from the bovine, porcine, biosynthetic, and fish skin groups in an embodiment. Although studies have provided, evidence of fish skin grafts being a preferred embodiment, a bovine, porcine, or biosynthetic skin could, of course, still be used as an effective skin substitute according to the present disclosure, and under some conditions or considerations, may also be a preferred embodiment of a skin substitute as contemplated in the current disclosure.

In an embodiment, the skin substitute, or skin graft material, is an acellular dermal matrix. The acellular dermal matrix is a biological scaffold-type material, such as a fish skin product, configured to be absorbed and grown into by the skin cells as the wound heals. The acellular dermal matrix may comprise decellularized, lyophilized fish skin.

In an embodiment, the acellular dermal matrix comprises lipids from a lipid layer of the decellularized fish skin to enhance the natural healing process of human skin. In a preferred embodiment, the skin substitute comprises an extracellular matrix product in a three-dimensional form of particles, a sheet, or a mesh to enhance structural support and improve tissue regeneration and cellular ingrowth.

Additionally, in an embodiment, the skin substitute is fenestrated. The fenestration may be defined as a slot extending through the skin substitute's thickness. In an embodiment, the fenestration is formed as a spiral. Providing the skin substitute with at least one fenestration allows for proper wound drainage of exudate during healing. It will be understood that in the context of the embodiments and methods for using the same, the terms fluid, moisture, and exudate are used interchangeably regarding wounds and wound dressings.

The skin substitute is in direct contact with a removable protective polymer substrate, i.e., a polymeric contact layer. The protective polymer substrate in an embodiment includes a hydrophobic membrane layer and an elastomeric layer. The wound treatment is configured to be applied to a wound such that the skin substitute is in contact with the wound, and the polymeric contact layer overlays the skin substitute, wherein the polymeric contact layer provides additional wound coverage.

The elastomeric layer is advantageously in direct contact with the skin graft material to hold the skin substitute in place while have gentle adhesive properties so that, after proper tissue regeneration and healing at the wound site, the elastomeric layer can conveniently separate from the skin substitute. The elastomeric layer is biocompatible with the skin and provides a gentle adhering bond to both the skin and skin substitute for proper fixation to the skin and painless separation after healing. The elastomeric layer has sufficient tackiness to negate a need for an adhesive between the elastomeric layer and the skin graft material.

The polymeric contact layer, comprising the elastomeric layer and hydrophobic membrane layer, is hydrophobic to allow for immersion of the wound treatment within a saline solution before application to the skin. Advantageously, the elastomeric layer retains its gentle adhesive properties after immersion. After application of the skin graft material to a wound, after some time has elapsed, e.g., preferably allowing for cellular ingrowth and tissue regeneration at the wound site, the elastomeric layer of the protective polymer substrate is configured to separate from the skin substitute conveniently. In an embodiment, the period may be for up to fourteen days or for a period or a time window that allows for cellular ingrowth and tissue regeneration at the wound site.

In an embodiment, the polymeric contact layer has a predetermined porosity to permit wound exudate to pass through its thickness. The predetermined porosity defines a distribution of apertures over a surface area of the polymeric contact layer. The distribution of apertures is configured to provide sufficient transfer of wound exudate while allowing sufficient contact between the surface area of the elastomeric layer and the skin to maintain a gentle adherence.

In an embodiment, the release liner comprises a polyolefin film. The release liner is hydrophobic to allow for immersion of the wound treatment within a saline solution before application to the skin. The release liner protects the skin and wound contacting surfaces of the elastomeric layer and skin substitute, respectively, from contamination before application of the wound treatment. The release liner allows the wound treatment to be handled in an aseptic manner.

In an embodiment, the release liner is transparent to allow for observation of the skin substitute and integrated polymeric contact layer. The release liner preferably comprises at least one flap to conveniently separate the release liner from both the skin substitute and elastomeric layer. Providing the release liner with flaps or a slit allows for the release liner to be easily grasped and pealed from the wound treatment before application to the wound. In an embodiment, the release liner comprises a first layer and a second layer, wherein the second layer overlaps a portion of the first layer of the release liner. The portion of the first layer is thus arranged between the second layer and the acellular dermal matrix.

The wound treatment is manufactured in an embodiment by integrating the skin substitute with the polymeric contact layer. The method of manufacturing the wound treatment includes harvesting a fish skin, decellularizing the fish skin, lyophilizing the decellularized fish skin, and cutting the lyophilized fish skin to a predetermined size. The step of integrating the decellularized, lyophilized fish skin with a polymeric contact layer, e.g., being a prefabricated silicone-coated hydrophobic membrane, includes disposing of the decellularized, lyophilized fish skin between the polymer contact layer and a peelable release liner.

A manufacturing tool opens the release liner just enough for the skin substitute to be placed on the elastomeric precisely and allows for easy re-assembling of the release liner without losing any adhesive properties or misplaced product. The peelable release liner is thus in direct contact with an elastomeric layer of the polymeric contact layer, and the decellularized, lyophilized fish skin is disposed of between the peelable release liner and the elastomeric layer.

Moreover, the polymeric contact layer protects the fish dermal matrix layer as additional wound coverage, and the elastomeric layer acts as a gentle adhesive contact layer to the skin surrounding the wound. However, the acellular dermal matrix is configured to be provided in direct contact with the elastomeric layer that has a gentle adhesion, as the elastomeric layer has sufficient tackiness to hold the acellular dermal matrix in position before application to the wound of a patient. During the removal of the polymeric contact layer, the acellular dermal matrix can remain on or integrated into the wound and can be absorbed by the wound during healing.

The method may further comprise packaging the assembled, integrated wound treatment within a package of synthetic flashspun high-density polyethylene fibers. The method may also comprise sterilizing the wound treatment. The whole wound treatment is, therefore, capable of being submerged in a sterile saline solution before being applied to a wound without losing its gentle adhesive properties. The integrated wound treatment may therefore be handled in an aseptic manner. After submersion in sterile saline solution, the release liner can be removed before applying the wound treatment to the wound.

The wettability of the wound treatment is a significant improvement over skin grafts and wound dressings of the prior art. The hydrophobic qualities of the polymeric contact layer, including the release liner, allow for submersion and hydration of the skin substitute without disrupting the tackiness or adhesion of the elastomeric layer. The wound treatment saves time for practitioners, avoids common securing methods of traditional adhesives over a skin graft, and allows a user to hydrate the device and subsequently place the device on the wound without considering supplemental fixation devices. The disclosed integrated wound treatment thus delivers fast and efficient wound care management for practitioners and patients.

Numerous other advantages and features of the present disclosure will become more readily apparent from the following detailed description, the accompanying examples, drawings, and the appended claims.

### BRIEF DESCRIPTION OF THE DRAWINGS

Features, aspects, and advantages of the presently disclosed technology may be better understood concerning the following description, appended claims, and accompanying drawings. A person skilled in the relevant art will understand that the features shown in the drawings are purposes of illustration, and variations, including different or additional features and arrangements thereof, are possible.

FIG. 1 is a perspective view of an embodiment of a wound treatment of the present disclosure.

FIG. 2 is a perspective view of a skin graft material used in the disclosed wound treatment.

FIG. 3 is a perspective view of a polymeric contact layer used in the disclosed wound treatment.

FIG. 4 is a plan view of the skin graft material integrated with the polymeric contact layer of an embodiment of the wound treatment.

FIG. 5 is a plan view of a release liner used in the disclosed wound treatment.

FIG. 6 is a sectional view of an embodiment of a wound treatment of the present disclosure.

FIG. 7 is a sectional view of the wound treatment applied to a wound.

FIG. 8 is a sectional view of removing the polymeric contact layer at the site of the wound.

FIG. 9 is a perspective view of an embodiment of a wound treatment of the present disclosure.

FIG. 10 is a perspective view of an alternative embodiment of a wound treatment of the present disclosure.

FIG. 11 is a perspective view of an alternative embodiment of a wound treatment of the present disclosure.

FIG. 12 is a perspective view of a skin graft material used in the embodiment of FIG. 11.

FIG. 13 is a plan view of the skin graft material used in the embodiment of FIG. 11.

FIG. 14 is an exemplary method of manufacturing an embodiment of a wound treatment of the present disclosure.

FIG. 15 is an exemplary method of assembling an embodiment of a wound treatment of the present disclosure.

The drawings are to illustrate exemplary implementations and are not drawn to scale. It is understood that the embodiments and methods for using the same are not limited to the arrangements and instrumentalities shown in the drawings.

### DEFINITIONS

Descriptions of the following terms are provided for further ease of understanding the embodiments of the disclosed wound treatment. As used herein, the term “wound treatment” means a multi-layered, integrated wound dressing. The term “wound dressing” means an interactive product (e.g., bandage, covering, compress, protective layer) that can manage a wound.

The term “skin substitute” means a group of elements or materials that enables the temporary or permanent occlusion of a wound. In a preferred embodiment, the skin substitute is an acellular dermal matrix, e.g., a biological scaffold-type material.

The terms “acellular,” “decellularized,” “decellularized fish skin,” and the like as used herein refer to a fish skin from which a substantial amount of cellular and nucleic acid content has been removed, leaving a complex three-dimensional interstitial structure of ECM. In embodiments, “decellularized fish skin” may further entail fish skin which, in addition to the complex three-dimensional interstitial structure of ECM, absent a substantial amount of cellular and nucleic acid content, includes omega 3 polyunsaturated fatty acids (PUFAs).

The terms “extracellular matrix” or “ECM”, as used herein, refer to the non-cellular tissue material present within the fish skin that provides structural support to the skin cells and performs various other important functions. The ECM described herein does not necessarily include matrix material constituted or re-formed entirely from extracted, purified, or separated ECM components (e.g., collagen). But in some embodiments, an ECM used as a skin substitute may include matrix material constituted or re-formed entirely from extracted, purified, or separated ECM components (e.g., collagen).

The term “treatment” is intended to be understood by its common dictionary definition. The term “treatment” broadly includes medical care devices (i.e., bandages and dressings) and/or medicaments given to a patient for an illness or injury. As should be appreciated by those with skill in the art, a “treatment” includes using a chemical, physical, or biological agent to preserve or give particular properties to something. Thus, a “treatment” may be the medical care provided (i.e., in the form of a method or series of prescribed acts), or it may refer to the medicament used to preserve or give a particular property to something.

The term “wound,” as used herein, is intended to encompass tissue injuries generally. Thus, the term “wound” includes those injuries that cause, for example, cutting, tearing, and/or breaking of the skin, such as lacerations, abrasions, incisions, punctures, avulsions, or other such injuries. Wounds may be described by any size, shape, or magnitude of the wound. For example, a paper cut is exemplary of a small, straight incision of relatively little magnitude, whereas a concussive blast resulting in a major laceration covering one or multiple body parts exemplifies a relatively larger wound of greater magnitude. However, each of the foregoing examples falls within the scope of the term “wound,” as used herein.

The term “wound” additionally includes damage to underlying tissue, such as that caused by traumatic injury. As such, the term “wound” is intended to include a combination of multiple different wounds. For example, a traumatic amputation caused by an explosive blast may generally be referred to as a wound, even though it is a compilation of a host of different lacerations, abrasions, avulsions, and punctures. Additionally, any underlying tissue damage resulting from the aforementioned explosive blast may further be encompassed within the understanding of this reference to a wound.

The term “wound” is also intended to encompass tissue injuries caused by burns (e.g., thermal and/or chemical burns). Further, the term “wound” is also intended to encompass injuries resulting from, for example, diabetic foot ulcers, venous leg ulcers, surgical operations, pressure ulcers, and other causes.

The term “biocompatible” refers to a material that is substantially non-toxic in the in vivo environment of its intended use and is not substantially rejected by the patient's physiological system (i.e., is non-antigenic).

The term “polymeric contact layer” is a polymeric covering impermeable to fluids and bacteria but allows moisture to permeate the covering.

The term “elastomeric layer” means a soft, medical-grade, gel-like layer designed to act as a gentle adhesive contact layer to the skin surrounding the wound. In a preferred embodiment, the elastomeric layer is silicone.

The term “fenestration” means a perforation, slit, slot, aperture, or opening that extends through the thickness of a layer.

Unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value, or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

It will also be noted that, as used in this specification and the appended claims, the singular forms “a,” “an,” and “the” do not exclude plural referents unless the context dictates otherwise. Thus, for example, an embodiment referencing a singular referent (e.g., “widget”) may also include two or more such referents.

Although the present disclosure will refer to particular embodiments, it is to be understood that these embodiments are merely illustrative of the principles and applications of the present disclosure. It will be apparent to those skilled in the art that various modifications and variations can be made to the disclosed wound dressing (i.e., wound dressing 100, 200, 300, 400) without departing from the scope of the present disclosure. Thus, where feasible, it is intended that the principles and aspects of each embodiment can apply to one another.

### DETAILED DESCRIPTION

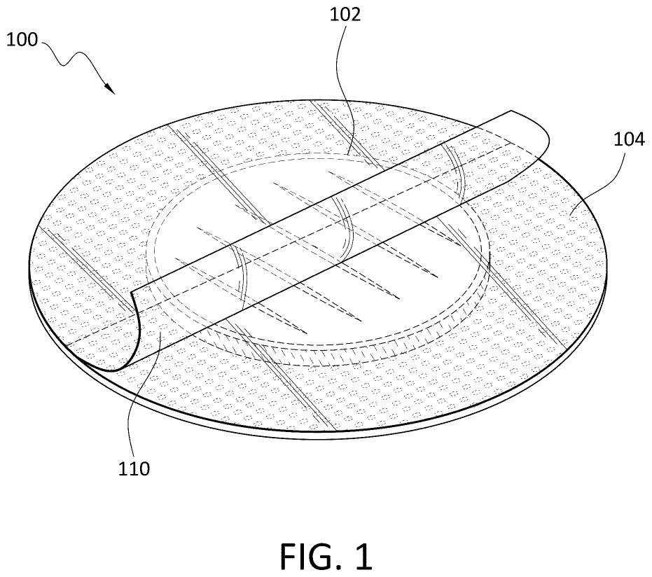

FIG. 1 depicts a wound treatment 100 according to the present disclosure. In an exemplary embodiment, the wound treatment 100 is a wound dressing comprising an intact skin substitute 102 used to support tissue regeneration of wounds and an integrated, adherent polymeric contact layer 104, which helps protect the wound surface and fix the skin substitute 102 in position. The wound treatment further comprises a release liner 110. The wound treatment 100 is suitable for the management of the following wounds: partial and full thickness wounds, pressure ulcers, venous ulcers, chronic vascular ulcers, diabetic ulcers, and trauma wounds (e.g., abrasions, lacerations, partial thickness burns, skin tears), and surgical wounds (e.g., donor sites/grafts, post-Mohs surgery, post-laser surgery, podiatric, wound dehiscence).

The wound treatment 100 is ideal for in-office applications and the integrated design allows for faster processing and handling, thus saving time for users. The wound treatment 100 can be completely submerged in a saline solution and does not require additional materials or devices for fixation at the wound site. The wound treatment 100 provides an efficient and simplified option for wound care management. The unique layers and relationships between layers of the wound treatment 100 are described in greater detail below.

Skin substitutes 102 may be considered broadly as a group of elements or materials that enable the temporary or permanent occlusion of a wound. Skin substitutes 102 can generally be divided into biological, synthetic, or hybrid skin substitutes, including biological and synthetic ones. Biological skin substitutes often have a more intact extracellular matrix structure, while synthetic skin substitutes can be synthesized on demand and modulated for specific purposes. Biological skin substitutes and synthetic skin substitutes each have advantages and disadvantages.

The biological skin substitutes may allow the construction of a more natural new dermis and allow excellent re-epithelialization characteristics due to the presence of a basement membrane. Synthetic skin substitutes may be chemically synthesized and provide the advantages of increased control over scaffold composition. Synthetic skin substitutes include synthetic biolayers, including, for example, a synthesized collagen or protein-based matrix or collagen or protein-based components combined with silicone components. Hybrid skin substitutes may be partly synthesized or produced by living cells and partly chemically synthesized.

Whether biological, synthetic, or hybrid skin substitutes are used, the object of using skin substitutes is to provide an effective, timely, and scar-free wound healing with as much return to the functions of the skin before the wound event. Examples of such skin substitutes are described in U.S. Patent Publication No. 2022/0313873, filed Mar. 24, 2022, and incorporated herein by reference in its entirety.

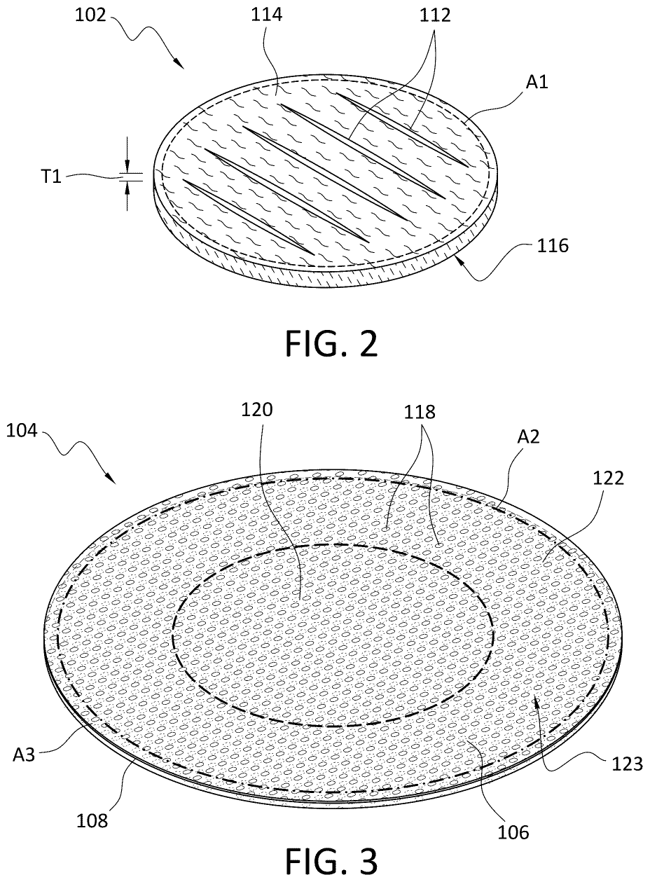

FIG. 2 depicts an exemplary skin substitute 102 according to the present disclosure. In an embodiment, the skin substitute 102 is an intact, decellularized fish skin product, such as one described in U.S. Pat. No. 8,613,957. The decellularized fish skin product is an intact scaffold that supports the ingrowth of endothelial and/or epithelial cells. Due to its porous scaffold design, the decellularized fish skin product is capable of 3-D cell ingrowth. The decellularized fish skin product is biocompatible and can thus be integrated by the host at the wound site. In an embodiment, the skin substitute 102 is made from the minimally processed skin of wild-caught Atlantic cod originating from Iceland. The fish skin is structurally alike to human skin with three basic layers including epidermis, dermis, and hypodermis and contains proteins, lipids, fatty acids, and other bioactive compounds that are homologous to human skin.

The fish skin embodiment of the skin substitute 102 is preferably treated with one or more decellularizing solutions to remove cellular material, including antigenic material, from the fish skin with minimal to no damage to the mechanical and structural integrity and biological activity of the naturally occurring extracellular matrix. The terms “extracellular matrix” or “ECM” refer to the non-cellular tissue material within the fish skin that provides structural support to the skin cells and performs various other important functions. The ECM product described herein does not include matrix material constituted or re-formed entirely from extracted, purified, or separated ECM components (e.g., collagen). According to one or more embodiments, the ECM product may be particles, a sheet, or a mesh.

The terms “acellular,” “decellularized,” “decellularized fish skin,” and the like as used herein refer to a fish skin product from which a substantial amount of cellular and nucleic acid content has been removed, leaving a complex three-dimensional interstitial structure of ECM. Decellularization disrupts the cell membranes and releases cellular content. Decellularizing may involve one or more physical treatments, one or more chemical treatments, one or more enzymatic treatments, or any combination thereof. This material is cost-effective, can be converted into a usable resource, and retains key biological features comparable to other animal-derived skin technologies.

In certain embodiments, decellularization (and other optional processing steps) does not remove all of the naturally occurring lipids from the lipid layer of the fish skin. Thus, the scaffold material can comprise one or more lipids from the fish skin, particularly from the lipid layer. The lipids in the scaffold material can include, for example, fatty acyls (i.e., fatty acids, their conjugates, and derivatives). In certain embodiments, the fatty acids include omega-3 fatty acids, such as eicosapentaenoic acid (EPA) and docosahexaenoic acid (DHA) (found in high concentrations in fish oil). The omega-3 fatty acids enhance tissue regeneration and the fish skin's ability to act as a bacterial barrier. The fish skin embodiment of the skin substitute 102 also provides the unique advantage of avoiding cultural and/or religious barriers to clinician and patient acceptance. Moreover, the fish skin embodiment of the skin substitute 102 is hypoallergenic, avoids the risk of disease transfer, and does not require multi-layer grafts. In an embodiment, the skin substitute 102 is selected from the bovine, porcine, biosynthetic, and fish skin groups.

The skin substitute 102 in FIG. 2 features fenestrations 112 to facilitate wound exudate drainage during healing. The fenestrations 112 may be designed as slots, apertures, or other openings that allow proper drainage through the thickness T1 of the skin substitute 102. Providing fenestrations 112 as slots allows for the flow of wound exudate through the skin substitute 102 and increases the flexibility of the dry wound treatment 100 while minimizing product loss and maximizing the contact area of the skin substitute 102 to the wound site.

In an embodiment, the skin substitute 102 comprises between 1 and 10 linear fenestrations 112, preferably five linear fenestrations. However, one skilled in the art will recognize that the design of the fenestrations may vary depending on the size of the skin substitute or application of the wound treatment 100. For example, the fenestrations may be curvilinear or circular.

In an embodiment, the skin substitute 102 is shaped like a disc and has wound contacting surface 114 with a surface area A1 between 2 square cm and 10 square cm. The skin substitute 102 may have a surface area A1 less than 2 square cm or greater than 10 square cm; however, it is preferred that the skin substitute 102 has a surface area A1 approximately equal to the wound size. The size of skin substitute 102 is non-limiting and may be produced, provided, or trimmed to fit the size and shape of the wound to be treated. Further, the skin substitute 102 can be configured as a meshed decellularized fish skin, particalized, comminuted, or otherwise processed into various sizes and shapes (square, rectangular, pentagonal, hexagonal, etc.).

FIG. 3 depicts an exemplary polymeric contact layer 104 per the present disclosure. In an embodiment, the polymeric contact layer 104 comprises a prefabricated hydrophobic membrane 108 coated with an elastomeric layer 106. The polymeric contact layer 104 is a non- or gentle-adherent wound contact layer. The polymeric contact layer 104 allows for painless removal from the skin. In a preferred embodiment, the polymeric contact layer 104 is non-adherent directly to the wound and exhibits soft adherence on peri-wound skin. The polymeric contact layer 104 is configured to remain in place, e.g., fixed in the same location on the skin as originally applied, for up to fourteen days. In an embodiment, the period may be for up to fourteen days, the lifetime approved duration of the device, or a duration that allows for cellular ingrowth and tissue regeneration at the wound site.

In an embodiment, the polymeric contact layer 104 comprises a plurality of apertures 118. The apertures 118 extend through the hydrophobic membrane 108 and elastomeric layer 106. In a preferred embodiment, the apertures 118 extending through the hydrophobic membrane 108 correspond or line up with the apertures 118 extending through the elastomeric layer. Thus, the apertures 118 continuously form through the polymeric contact layer 104.

In an embodiment, the apertures 118 are provided as circular perforations to allow for wound exudate to flow freely from the wound site through the polymeric contact layer 104. In an embodiment, the diameter of the apertures is between 0.5 mm and 5 mm, preferably between 1 mm and 4 mm.

In an embodiment, the polymeric contact layer 104 has a predetermined porosity to permit wound exudate to pass through the thickness of the polymeric contact layer 104. The predetermined porosity defines a distribution of apertures 118 over a surface area of the polymeric contact layer 104. The distribution of apertures 118 is configured to provide sufficient transfer of wound exudate while allowing sufficient contact between the surface area of the elastomeric layer and the skin to maintain a gentle adherence.

Distribution or porosity of the apertures 118 over the surface area of the elastomeric layer 106 is between 5% and 40%, preferably between 10% and 35%. In a preferred embodiment, the distribution of apertures is less than 25%, e.g., approximately 17%. Preferably, the distribution of the apertures 118 over the surface area of the polymeric contact layer 104 is porous enough without compromising the sheet-like nature of the skin substitute 102.

In an embodiment, the polymeric contact layer 104 comprises a central portion 120 on the elastomeric layer 106 configured to directly engage with a silicone contacting surface 116 the skin substitute 102. The surrounding portion of the polymeric contact layer 104 outside the central portion 120 defines a perimeter portion 122 configured to directly engage with the peri-wound skin. The surface area A2 of the elastomeric layer 106 is substantially equal to the surface area A3 of the hydrophobic membrane 108.

In an embodiment, the polymeric contact layer 104 is shaped like a disc, wherein the elastomeric layer 106 and hydrophobic membrane 108 have surface areas A2 and A3 between 4 square cm and 20 square cm. The surface areas A2, A3 may be less than 4 square cm or greater than 20 square cm. It is preferred that the ratio of surface areas A2, A3 to the surface area A1 of the skin substitute 102 is 2:1, 3:1, 3:2, 4:1, 4:3, 5:1, 5:2, 5:3, or 5:4. One skilled in the art will recognize that the ratios of the surfaces areas A2, A3 to surface area A1 can be varied depending on the application; however, the surface area A2 should be sufficiently greater than and sufficiently encompasses the surface area A1 so that the polymeric contact layer 104 remains in place and contact with the peri-wound skin for the desired duration.

In an embodiment, the elastomeric layer 106 has a gentle adhesion between 0.015 and 0.85 N/cm, preferably between 0.035-0.8 N/cm. The elastomeric layer 106 is a hydrophobic, moisture-impervious layer bonded to the hydrophobic membrane 108 to form an integrated polymeric contact layer 104. This is advantageous for the skin substitute 102, which may need to be hydrated before use. Therefore, the wound treatment 100 is handled in an aseptic manner, arranged to be submerged in a sterile saline solution for rehydration (or hydration) before being applied to a wound and without losing its gentle adhesive properties. The elastomeric layer 106 is preferred over alternative adhesives and polymers because it prevents microbial growth and dermatitis.

Additionally, the biocompatible properties of the elastomeric layer 106 demonstrate low thermal conductivity, low chemical reactivity, and low toxicity. In an embodiment, the elastomeric layer 106 is preferred to be transparent for observing the peri-wound skin and passage of exudate through the corresponding apertures 118. In an alternative embodiment, the elastomeric layer 106 is colored or skin-toned to provide an aesthetically pleasing appearance or noticeable indication of the wound site. In an alternative embodiment, the elastomeric layer 106 comprises both transparent and colored or skin-toned sections to allow for both observation of the wound site and aesthetic appeal.

The hydrophobic membrane 108 is an elastic, moisture-impervious barrier bonded to the elastomeric layer 106. The hydrophobic membrane 108 augments the elastomeric layer 106 to improve durability and extend the lifetime, or use, of the wound treatment 100. The hydrophobic membrane 108 is permeable to gases and water vapor and impermeable to proteins and bacteria. The outer surface 123 of the hydrophobic membrane 108, which is the side of the hydrophobic membrane 108, is not bonded to the elastomeric layer 106 and acts as a protective layer for the polymeric contact layer 104.

The hydrophobic membrane 108 is resistant to mechanical wear, reduces infection rates, and maintains ideal wound moisture. In an embodiment, it is preferred that the hydrophobic membrane 108 is transparent for observing the peri-wound skin and passage of exudate through the corresponding apertures 118. In an alternative embodiment, the hydrophobic membrane 108 is colored or skin-toned to provide an aesthetically pleasing appearance or noticeable indication of the wound site. In an alternative embodiment, the hydrophobic membrane 108 comprises both transparent and colored or skin-toned sections to allow for both observation of the wound site and aesthetic appeal.

The hydrophobic membrane 108 includes a biocompatible polymer. Any polymer materials usable as dressing material may be used as biocompatible polymers without limitation and may be properly chosen by those skilled in the art. The biocompatible polymer may, for example, include one or more: polyvinyl alcohol, polyurethane, polyethylene, polyethylene oxide, low-density polyethylene, polyacrylic acid, polyoxyethylene, polytetrafluoroethylene, polypropylene, polyethylene terephthalate, polyamide, polyacrylonitrile, polyester, polyvinyl chloride, polyvinylidenefluoride, polysiloxane (a silicone rubber), polyglycolic acid, polylactic acid, polymethacrylic acid, polyacrylamide, polysaccharide, polyvinylpyrrolidone, silicone, alginic acid, sodium alginate, cellulose, pectin, chitin, chitosan, gelatin, collagen, fibrin, hyaluronic acid, natural rubber, synthetic rubber, or combinations thereof. In a preferred embodiment, the hydrophobic membrane 108 includes polyurethane.

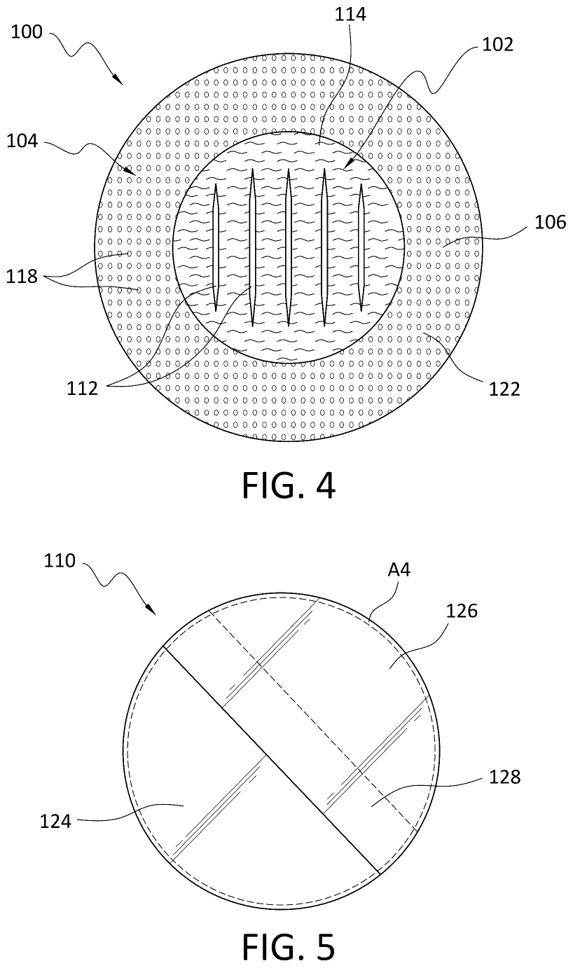

FIG. 4 depicts the wound treatment 100 comprising the skin substitute 102 and the polymeric contact layer 104. The skin substitute 102 is in direct contact with the elastomeric layer 106 of the polymeric contact layer 104, wherein the border(s) of the skin substitute 102 are encompassed by the border(s) of the polymeric contact layer 104. Specifically, the skin substitute 102 is disposed of within the perimeter portion 122 of the polymeric contact layer 104.

In an embodiment, the distributional area of fenestrations 112 over the surface area A1 of the skin substitute 102 is between 0% and 49% compared with the distributional area of the apertures 118 over the surface area A2 of the elastomeric layer 106. The apertures 118 through both the hydrophobic membrane 108 and elastomeric layer 106 allow for a sterile saline solution to pass through the thickness of the polymeric contact layer 104 and saturate the skin substitute 102 without compromising the gentle adhesive properties of the elastomeric layer 106.

FIG. 5 depicts an exemplary release liner 110 that may be used by the disclosed wound treatment 100. The release liner 110 acts as a pre-application protective layer that is placed over and in direct contact with both the elastomeric layer 106 and the skin substitute 102 such that it protects and shields the elastomeric layer 106 and skin substitute 102 from contamination before being applied to the wound. The release liner 110 is made of a material and provided on the wound treatment 100 in such a way as to be easily removed by, for example, a medical practitioner before the wound treatment 100 is applied to the wound. In other words, the release liner 110 is non-adherent to the skin substitute 102.

In an embodiment, the release liner 110 includes at least one flap to facilitate the removal of the release liner 110 from the elastomeric layer 106 and skin substitute 102. In an embodiment, the surface area A4 of the release liner 110 is substantially equal to or slightly greater than the surface area of the elastomeric layer 106. This allows adequate protection and coverage for the skin substitute 102 and the elastomeric layer 106.

In an embodiment, the release liner 110 comprises a polyolefin film material. Polyolefin is preferred because it is generally considered more environmentally friendly than other films due to its low material usage and recyclability. Other suitable polymers for the release liner 110 include, but are not limited to, low-density polyethylene, polyvinyl alcohol, nylon, polyester, polystyrene, polymethylpentene, polyoxymethylene, copolymers thereof, and mixtures thereof. The material of the release liner 110 is chosen so that the release liner 110 can be removed from both the skin substitute 102 and the elastomeric layer 106 without disrupting the material properties of the wound treatment 100.

The release liner 110 remains in contact with the skin substitute 102 and the elastomeric layer 106 while submerged in a sterile saline solution. Other suitable materials may be used for the release liner 110, and the material(s) of the release liner 110 should be selected not to decrease the adhesive qualities of the elastomeric layer 106 when the release liner 110 is removed from the wound treatment 100. In an embodiment, the release liner 110 is biodegradable.

In an embodiment, the release liner 110 comprises a first sheet 124 and a second sheet 126 that overlap and form portions that cover the surface area A4 of the release liner 110. An overlapping region 128 is formed by the section of the second sheet 126 that overlaps with the first sheet 124. The overlapping region 128 allows users to easily grasp and peel apart the first and second sheets 124 and 126 away from the skin substitute 102 and the elastomeric layer 106 before the wound treatment 100 is applied to the wound. In an embodiment, the first sheet 124 is substantially equal to the second sheet 126 in size and shape. In an alternative embodiment, the first sheet 124 is greater than the second sheet 126 in size and shape, wherein the overlapping region 128 is provided at a minor arc or closer to a peripheral edge (depending on shape) of the release liner 110.

In a preferred embodiment, regardless of the presence of an overlapping region 128, the total surface area coverage provided by the first and second sheets 124, 126 release liner 110 is greater than or equal to the surface area of the elastomeric layer 106 to provide appropriate protection of the skin substitute 102 and elastomeric layer 106. In an embodiment, the release liner 110 is provided with a slit (e.g., non-overlapping region) that divides the release liner 110 into first and second sheets 124, 126, wherein the first and second sheets 124, 126 can be removed by users using aseptic techniques (i.e., pliers, pincers, forceps). Advantageously, the skin substitute 102, being disposed between polymeric contact layer 104 and the release liner 110, is arranged to be submerged in a saline solution, together with the polymeric contact layer 104 and release liner 110, for hydration of the skin substitute 102.

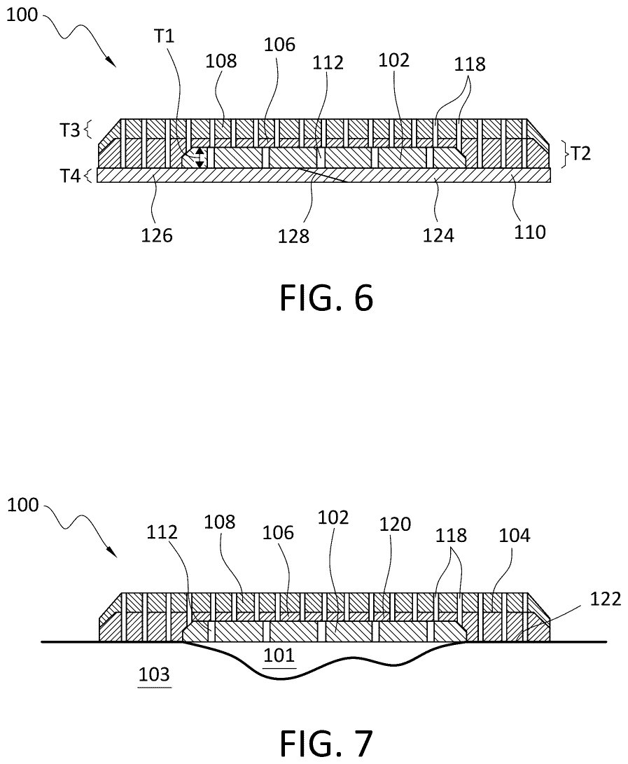

FIG. 6 shows a sectional view of an embodiment of the wound treatment 100. The skin substitute 102 has a first thickness T1. Generally, the first thickness is about 0.1 to 4.0 mm thick (i.e., in cross-section), such as 0.5, 1.0, 1.5, 2.0, 2.5, 3.0 or 3.5 mm thick. The thickness can depend on a number of factors, including the species of fish used as the starting material, processing, lyophilization, and rehydration, as discussed in greater detail in U.S. Pat. No. 8,613,957. In an embodiment, the first thickness T1 may comprise a skin substitute 102 having multiple layers. In one embodiment, the first thickness T1 is uniform across the surface area A1 of the skin substitute 102. In an alternative embodiment, the first thickness may taper at the edge(s), defining the surface area A1 of the skin substitute 102.

The polymeric contact layer 104 comprises a second thickness, T2, defining the gsm thickness of the elastomeric layer 106, and a third thickness, T3, defining the thickness of the hydrophobic membrane 108. The combined thickness of the polymeric contact layer 104 (e.g., the cross-sectional sum of the second thickness T2 and the third thickness T3) is preferably greater than 0.2 mm. Generally, the second thickness T2 is from about 50 to 200 gsm, such as 100, 110, 120, 130, 140, 150, 160, 170, 180, or 190 gsm thick. “GSM,” or Grams per Square Meter, measures the thickness of materials like paper and fabric. The gsm thickness T2 depends on the weight of the elastomeric (e.g., silicone) gel used in the elastomeric layer 106 and the surface area A2 of the elastomeric layer 106. In an embodiment, the second thickness T2 is uniform across the surface area A2 of the elastomeric layer 106. In an alternative embodiment, the second thickness T2 tapers at the edge(s), defining the surface area A2 of the elastomeric layer 106. In an embodiment, the second thickness T2 is reduced at the central portion 120 of the polymeric contact layer 104 to receive and partially contain the skin substitute 102 within a recess.

Generally, the third thickness, T3, is from about 25 to 100 μm, such as 50, 60, 70, 80, or 90 μm thick. The third thickness T3 is selected so that the hydrophobic membrane 108 is sufficiently pliable and elastic to conform to the skin around the wound site and exhibits sturdiness and strong resistance to humidity and organic solvents. In an embodiment, the third thickness T3 is uniform across the surface area A2 of the elastomeric layer 106. In an alternative embodiment, the third thickness T3 tapers at the edge(s), defining the surface area A3 of the hydrophobic membrane 108.

The release liner 110 comprises a fourth thickness T4, defining the gsm thickness of a pre-application protective layer. In an embodiment of a release liner 110 comprising first and second sheets 124, 126, the cross-sectional thicknesses of the first and second sheets 124, 126 are equal to the fourth thickness T4. Generally, the fourth thickness T4 is from about 50 to 100 gsm, such as 55, 65, 75, 85, or 95 gsm. The gsm thickness T4 depends on the weight of the material used for the release liner 110 and the surface area A4 of the release liner 110. In an embodiment, the fourth thickness T4 is uniform across the surface area A4 of the release liner 110. In an embodiment, the fourth thickness T4 is increased at the overlapping region 128, wherein the thicknesses of the first sheet 124 and the second sheet 126 overlap.

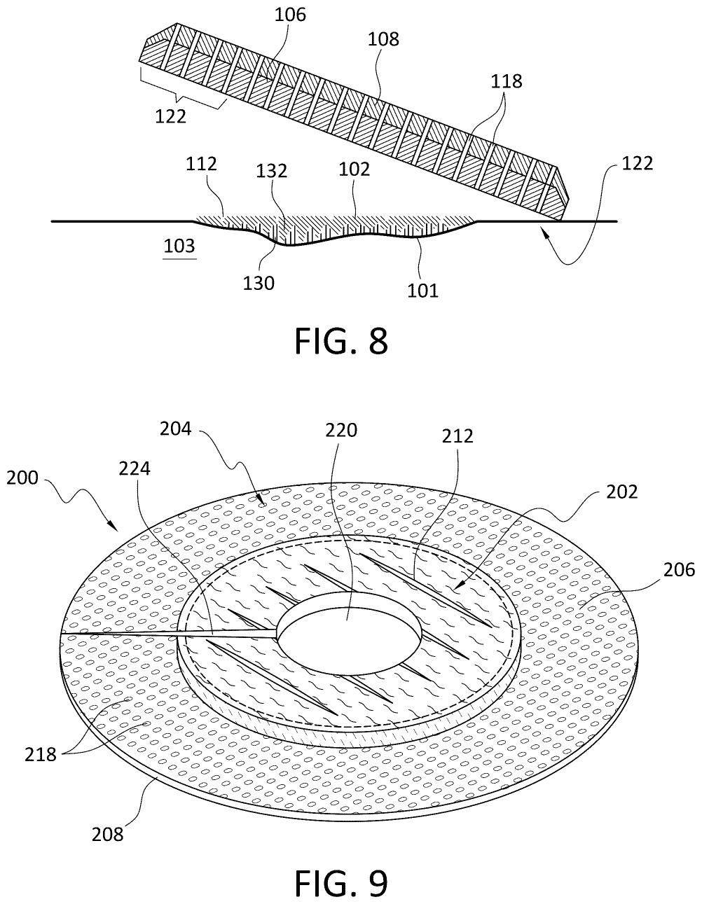

FIGS. 7 and 8 depict sectional views of the wound treatment 100 applied to and removed from a wound 101. As observed in FIG. 7, the skin substitute 102 is placed over the wound 101. The skin substitute 102 is aligned with the central portion 120 of the polymeric contact layer 104 and roughly fits or covers the wound 101 so proper healing can occur. The skin substitute 102 is provided in direct contact with the elastomeric layer 106 and has a gentle adhesion. The polymeric contact layer 104 is positioned so that the perimeter portion 122 of the polymeric contact layer 104 gently adheres to the skin 103 surrounding the wound 101.

The polymeric contact layer 104 protects the skin substitute 102, as additional wound coverage, and the elastomeric layer 106 acts as a gentle adhesive contact layer to the skin 103 surrounding the wound 101. The elastomeric layer 106 has sufficient tackiness to hold the skin substitute 102 in position (e.g., aligned with the central portion 120) before and during the wound treatment 100 application. Sufficient tackiness is defined as an adhesion between 0.015 and 0.85 N/cm, preferably between 0.035-0.8 N/cm.

FIG. 8 shows the removal of the polymeric contact layer 104 (including the elastomeric layer 106 and hydrophobic membrane 108) at the site of the wound 101. The perimeter portion 122 of the polymeric contact layer 104 does not interfere with the wound 101 and is easily and painlessly removed from the skin 103. During removal, and after healing has occurred, the polymeric contact layer 104 is removed from the skin 103, while the skin substitute 102 remains in contact with the healing wound 101.

In an embodiment wherein an acellular dermal matrix (e.g., decellularized fish skin) is used as the skin substitute 102, the skin substitute 102 supports cellular ingrowth 132 of endothelial and/or epithelial cells. As noted above, due to a porous scaffold design, the decellularized fish skin product is capable of 3-D cellular ingrowth 132. The enhanced protective support offered by the polymeric contact layer 104, in addition to the facilitated egress of wound exudate from the apertures 118, also supports healing to allow regenerated tissue 130 to form at the wound 101.

In another embodiment, a wound treatment 200 is provided to support wound healing in ostomy applications, wherein a surgically created hole (stoma) is cut through the skin of a patient. In these applications, it is beneficial to structurally reinforce the opening at the wound site and support an inserted transcutaneous device (e.g., tracheostomy tube). It is to be understood that wound treatment 200, described in FIG. 9, is an alternative embodiment of wound treatment 100, wherein aspects of wound treatment 100 can apply to wound treatment 200. FIG. 9 depicts a wound treatment 200 of the present disclosure comprising a skin substitute 202 and a polymeric contact layer 204. The polymeric contact layer 204 comprises an elastomeric layer 206 for directly contacting the skin around the wound or ostomy site and a hydrophobic membrane 208 to provide protection for skin substitute 202 and as additional wound coverage.

The polymeric contact layer 204 preferably comprises apertures 218 to promote proper drainage of wound exudate. The wound treatment 200 comprises an opening 220 that extends through the layers of skin substitute 202 and polymeric contact layer 204. The opening 220 allows for introducing a transcutaneous device into the ostomy site while still allowing the skin substitute 202 surrounding the wound site to promote healing.

In an embodiment, the skin substitute 202 comprises one or more fenestrations 212 to allow for proper wound drainage of exudate during healing. In an embodiment, the wound treatment 200 comprises a slit 224 that extends from the opening 220 to an outer edge of the polymeric contact layer 204. The slit 224 enables the wound treatment 200 to be flexibly positioned about an existing transcutaneous device that has been inserted at the ostomy site.

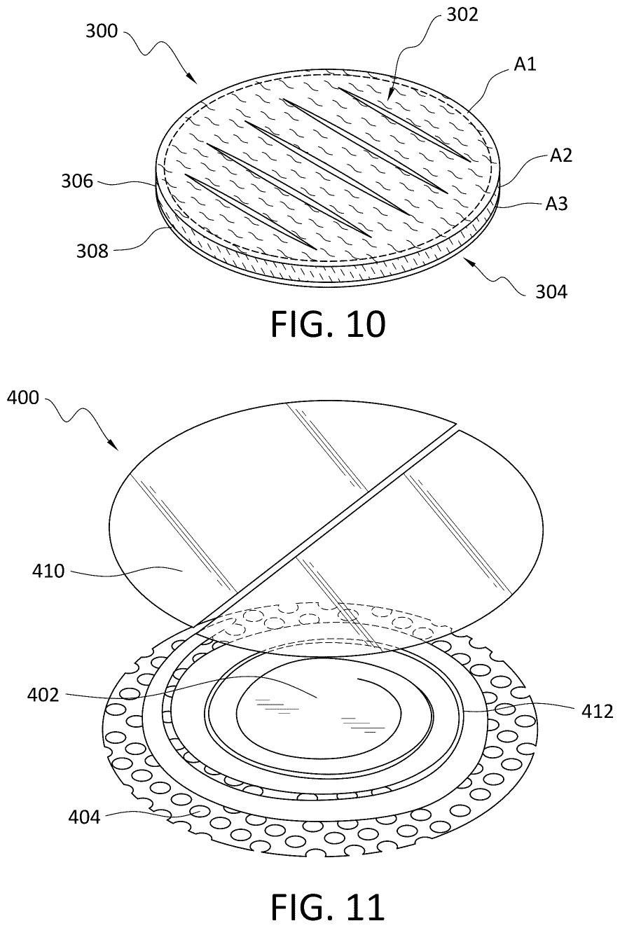

FIG. 10 depicts an alternative embodiment of a wound treatment 300 combined with a secondary dressing (e.g., a bandage). It is to be understood that the wound treatment 300 described with FIG. 10 is an alternative embodiment of the wound treatment 100, wherein aspects of the wound treatment 100 can apply to the wound treatment 300. The wound treatment 300 comprises a skin substitute 302 integrated with a polymeric contact layer 304. The polymeric contact layer 304 preferably comprises an elastomeric layer 306 in direct contact with the skin substitute 302 and a hydrophobic membrane 308 to protect skin substitute 302 and act as additional wound coverage.

In an embodiment, the surface area A1 of the skin substitute 302 equals the surface areas A2, A3 of the elastomeric layer 306 and hydrophobic membrane 308. In other words, the layers of the skin substitute 302 and polymeric contact layer 304 are the same shape and size. The polymeric contact layer 304 advantageously supports the wound treatment 300 for maintaining moisture at the wound site. In an embodiment, the skin substitute 302 comprises fenestrations, and the polymeric contact layer 304 comprises apertures to allow for proper wound drainage of exudate during healing.

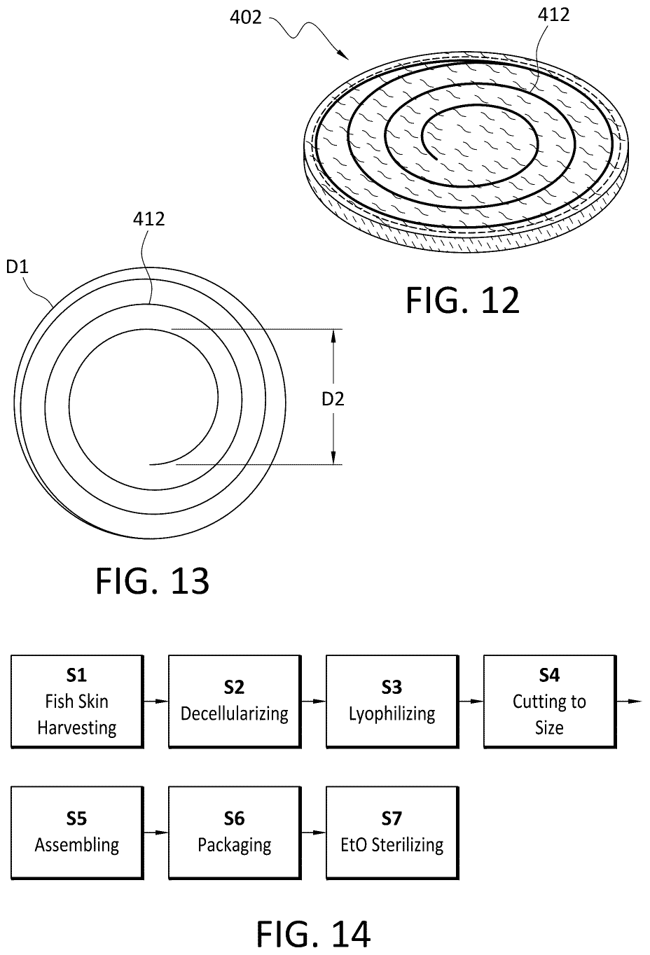

FIG. 11 illustrates an embodiment of a wound treatment 400 comprising a skin substitute 402, polymeric contact layer 404, and release liner 410. It is to be understood that the wound treatment 400 described with FIG. 11 is an alternative embodiment of the wound treatment 100, wherein aspects of the wound treatment 100 can apply to the wound treatment 400. The skin substitute 402, also depicted in FIG. 12, includes a spiral fenestration 412 with a spiral configuration extending through the thickness of the skin substitute 402. In an embodiment, the fenestration 412 is perforated to allow guided separation of the skin substitute 402 in a spiral pattern. The fenestration 412 further facilitates wound exudate drainage during healing. Providing the fenestration 412 as a spiral slot allows for the flow of wound exudate through the skin substitute 402, allows for quicker adjustment in size, and increases the flexibility of the dry wound treatment 400 while both minimizing product loss and maximizing the contact area of the skin substitute 402 to the wound site.

FIG. 13 depicts an exemplary spiral configuration for the fenestration 412 of the skin substitute 402. The skin substitute 402 can be conveniently trimmed from a first size or diameter D1 to a second size or diameter D2 by unwinding and cutting (or tearing) the skin substitute 402. The spiral configuration of the fenestration 412 allows for steady, controlled tailoring of the skin substitute 402 to accommodate various wound sizes. The fenestration 412 may be configured as an Archimedean spiral, logarithmic spiral, parabolic spiral, or Dürer spiral.

In an embodiment, the skin substitute 402 may have a first diameter D1 of 30 mm graft, wherein the fenestration 412 features 2 mm outer spiral sections to allow for trimming of the skin substitute to a second diameter D2. The second diameter D2 may be a minimum diameter, e.g., 15 mm. The spiral configuration of the fenestration 412 may be marked with indicia to inform the user of an approximate diameter and/or where to cut the skin substitute. Advantageously, the spiral fenestration 412 minimizes area loss and offers more flexibility in the customization of the skin substitute 402.

FIG. 14 depicts an exemplary method of manufacturing an embodiment of the disclosed wound treatment 100, 200, 300, 400. In a preliminary step S1, fresh fish skin is harvested. The fish used for harvesting fish skin are preferably wild-caught North Atlantic cod because no known viral transfer risk exists between North Atlantic Cod and humans. Next, the fish skin is decellularized in step S2 and lyophilized in step S3, using a method like that disclosed in greater detail in U.S. Pat. No. 8,613,957, to form a skin substitute.

The skin substitute is subsequently cut to a predetermined size in step S4. The predetermined size is established to correspond with a paired polymeric contact layer such that the paired polymeric contact layer encompasses the corresponding fish skin substitute. The polymeric contact layer is pre-cut and is manufactured to comprise a hydrophobic membrane, elastomeric layer, and release liner. The skin substitute is subsequently processed and assembled in step S5 in between the release liner and the elastomeric layer of the polymeric contact layer. In a preferred embodiment, an assembling tool partly peels the release liner from the elastomeric layer. It deposits the skin substitute at an acceptable position in direct contact with the elastomeric layer and release liner. The assembling tool closes the release liner to enclose the skin substitute against the elastomeric layer, thus resulting in an integrated wound treatment.

After assembling the integrated wound treatment, the wound treatment may be packaged in step S6 within a package of synthetic flashspun high-density polyethylene fibers (e.g., Tyvek) or other suitable packaging material. A final step in manufacturing the wound treatment includes appropriate sterilization techniques, such as using ethylene oxide (EtO) gas sterilization, in step S7.

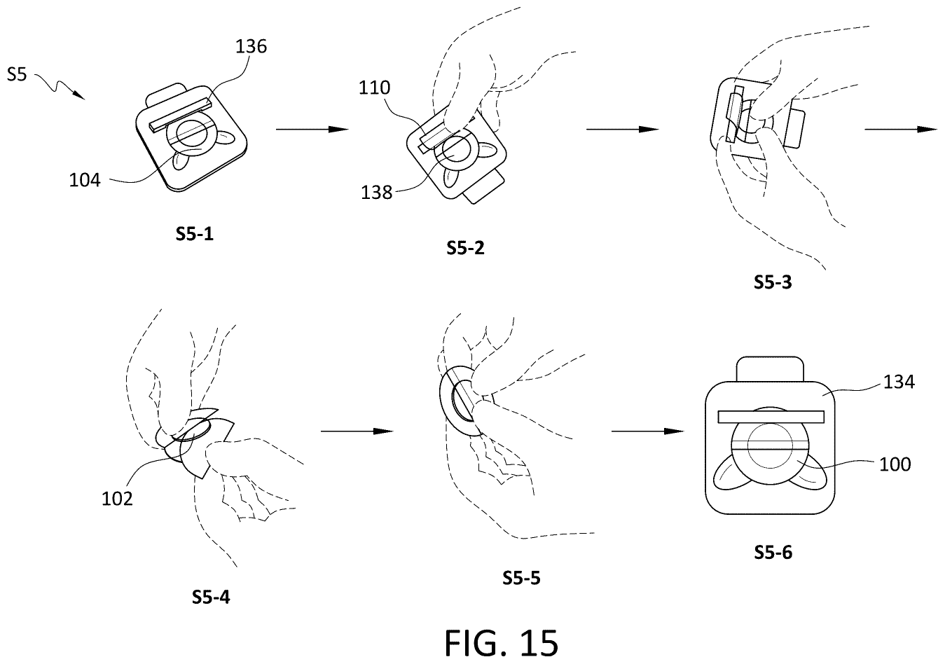

FIG. 15 depicts an exemplary method of assembling an embodiment of the disclosed wound treatment 100, 200, 300, 400. After removing the pre-cut polymeric contact layer 104 from its previously manufactured packaging, the polymeric contact layer 104, integrated with the release liner 110, is placed in an assembling tool 134. The assembling tool 134 comprises a slidable clamp 136 for holding the polymeric contact layer 104 during assembly. In the first assembly step S5-1, the assembly tool 134 engages with the release liner 110 by sliding the slidable clamp 136 over the overlapping second sheet 126 of the release liner 110 to hold the polymeric contact layer 104 in place.

The top side of the overlapping second sheet 126 of the release liner 110 is on the side of the assembling tool 134 that comprises the slidable clamp 136. Next, in a second assembling step S5-2, the overlapping second sheet 126 of the release liner 110 is pulled back until the side of the polymeric contact layer 104 receiving the skin substitute 102 is accessible. The liner 110 is held against the slidable clamp 136 during this step. During the third assembling step S5-3, the polymeric contact layer 104 is held in place by the slidable clap 136. At the same time, the skin substitute 102 is inserted to interface with the elastomeric layer 106 of the polymeric contact layer 104.

The skin substitute 102 is preferably attached to the elastomeric layer 106 using a centering element 138 of the assembling tool 134. In an embodiment, the centering element 138 is a circular element corresponding to the center of the wound treatment 100. The skin substitute 102 is attached to the elastomeric layer 106 using adequate pressure for the elastomeric layer 106 to stick to the skin substitute 102 without damaging the skin substitute 102. The slidable clamp 136 may then be slid away from the polymeric contact layer 104 to disengage with the wound treatment 100.

During step S5-4, the other first sheet 124 of the release liner 110 is subsequently maneuvered away from the polymeric contact layer 104 so that a remaining portion of the skin substitute 102 can interface with the elastomeric layer 106. It is preferred that the elastomeric layer 106 is not under tension (e.g., being pulled) while the skin substitute 102 is placed on the elastomeric layer 106. The first sheet 124 and second sheet 126 of the release liner 110 are then arranged back into an original overlapping position, wherein the second sheet 126 overlaps the first sheet 124. In step S5-5, adequate force is applied about the perimeter portion 122 of the polymeric contact layer 104 to reinforce the adhesive bond between the release liner 110 and the elastomeric layer 106. The assembled wound treatment 100 may then be placed back onto the assembling tool 134 to identify the correct placement of the skin substitute 102 in steps S5-6. Acceptable wound treatment 100 products are identified using the centering element 138 of the assembling tool 134.

It will be understood that the above-described embodiments of the disclosure may assume various shapes, sizes, and configurations without departing from the scope of the disclosure.

It will be understood that the above-described embodiments are illustrative in nature and that modifications thereof may occur to those skilled in the art. Accordingly, this disclosure is not to be regarded as limited to the embodiments disclosed herein, but is to be limited only as defined in the appended claims.

1. A wound dressing comprising:
skin substitute;
a polymeric contact layer including an elastomeric layer bonded to a hydrophobic membrane; and
a release liner arranged over and in contact with both the skin substitute and elastomeric layer;
wherein the skin substitute is located between the polymeric contact layer and the release liner;
wherein the skin substitute is arranged to be submersed for hydration, together with the polymeric contact layer and the release liner, in a saline solution.

2. The wound dressing of claim 1, wherein the skin substitute is an acellular dermal matrix comprising decellularized fish skin.

3. The wound dressing of claim 2, wherein the skin substitute comprises lipids from a lipid layer of the decellularized fish skin.

4. The wound dressing of claim 1, wherein the skin substitute comprises an extracellular matrix product in a three-dimensional form of particles, or a sheet, or a mesh.

5. The wound dressing of claim 1, wherein the skin substitute is in direct contact with the elastomeric layer of the polymeric contact layer.

6. The wound dressing of claim 1, wherein the skin substitute includes at least one fenestration that extends through a thickness of the skin substitute.

7. The wound dressing of claim 6, wherein the fenestration is formed as a spiral.

8. The wound dressing of claim 1, wherein the elastomeric layer has sufficient tackiness to negate a need for an adhesive between the elastomeric layer and the skin substitute.

9. The wound dressing of claim 1, wherein the elastomeric layer includes a silicone material and the hydrophobic membrane includes a polyurethane material.

10. The wound dressing of claim 1, wherein polymeric contact layer has a distribution of apertures defining a predetermined porosity over a surface area of the polymeric contact layer.

11. The wound dressing of claim 10, wherein the distribution of apertures is less than 25% of the contact layer surface area.

12. An integrated wound treatment comprising:
a skin substitute in direct contact with a polymeric contact layer, the polymeric contact layer including a hydrophobic membrane and an elastomeric layer, the elastomeric layer being in direct contact with the skin substitute;
wherein the elastomeric layer has sufficient tackiness to negate a need for an adhesive between the elastomeric layer and the skin substitute;
wherein the integrated wound treatment is configured to be applied to a wound such that the skin substitute is in contact with the wound and the polymeric contact layer overlays the skin substitute and further adheres to skin surrounding the wound;
wherein after application of the skin substitute to the wound, and following a time period that allows for cellular ingrowth and tissue regeneration at the wound with the skin substitute, the polymeric contact layer is arranged to separate from the skin substitute.

13. The integrated wound treatment of claim 12, wherein the polymeric contact layer has a predetermined porosity to permit wound exudate to pass through.

14. The integrated wound treatment of claim 12 wherein the skin substitute is fenestrated.

15. The integrated wound treatment of claim 12 wherein the skin substitute is selected from the group consisting of bovine, porcine, biosynthetic, and fish skin.

16. The integrated wound treatment of claim 12, wherein the skin substitute is an acellular dermal matrix comprising decellularized extracellular matrix from fish skin.

17. The integrated wound treatment of claim 12, wherein the integrated wound treatment includes a release liner in contact with the skin substitute, the skin substitute being provided between the release liner and the polymeric contact layer.

18. A method for producing an integrated wound treatment, the method comprising:
harvesting a skin substitute;
cutting the skin substitute to size; and
integrating the skin substitute with a polymeric contact layer;
wherein the polymeric contact layer includes hydrophobic membrane coated with an elastomeric layer;
wherein a release liner is arranged against the elastomeric layer of the polymeric contact layer; and
wherein the skin substitute is located between the release liner and the elastomeric layer.

19. The method of claim 18, wherein the skin substitute is a fish skin and comprises the following steps after harvesting the skin substitute:
decellularizing the fish skin; and
lyophilizing the decellularized fish skin.

20. The method of claim 19, further comprising sterilizing the integrated wound treatment using ethylene oxide gas.
